(19) United States
(12) Patent Application Publication
(10) Pub. No.: US 2020/0387212 A1
(43) Pub. Date: Dec. 10, 2020

(54) Systems and Methods of Interactive Virtual Reality Simulation

(71) Applicant: TechieChick IP, LLC

(72) Inventor: JESSICA MACKEY, Montgomery, TX (US)

(21) Appl. No.: 16/433,700

(22) Filed: Jun. 6, 2019

(51) Int. Cl. G06F 3/01; G06N 20/00; G09B 9/00; A61M 21/00

(52) U.S. Cl. CPC G06F 3/011; G06N 20/00; G06F 3/016; G09B 9/00; A61M 2021/005; A61M 2021/0016; A61M 2021/0022; A61M 2021/0027; A61M 21/00

(57) ABSTRACT

A system in a virtual reality environment that includes a computer system, an interactive hardware device where the interactive hardware device emulates an object in the virtual reality environment, and software residing in the computer system. The software includes an adaptive self-learning personality profile that is based on at least one of a user's inputs to the interactive hardware device and a predetermined set of parameters. The software also includes a character interface, where the character interface interacts with the user using the interactive hardware device based on the personality profile, where the personality profile is based at least in part on signals from the interactive hardware device, and where the personality profile generates responses to the user by sending signals to the interactive hardware device.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of virtual reality (VR), specifically for forming a VR interactive simulation environment using interactive hardware devices. Furthermore, the VR simulation uses personality profiles to emulate a human being to allow both predictable and unpredictable human behavior patterns to control the interactive hardware devices, thus providing for a dynamic and ever-changing experience. This disclosure can be used for training and simulation in the fields of medicine, underwater environments, adult entertainment, space exploration, and the like where it is too expensive or unrealistic to have a human present in the actual physical environment.

### Background of the Invention

In many environments, it is uncomfortable, difficult, expensive, or dangerous to place a user in an environment for training or simulation. As an example, astronauts practice maintenance procedures and working with other astronauts in a swimming pool to simulate a weightless environment as it is very expensive to practice in space. Another example is during a medical procedure where the patient may not react as expected, or where a new and unplanned event occurs, requiring the medical practitioners to react to a different set of problems. Yet another area is for the treatment of patients affected with sexually-related mental disorders, where actual physical contact with another human is unrealistic or not possible. In another example, it is advantageous for a user to simulate a conversation with another human prior to an actual face-face meeting such as during a job interview or a high-pressure social event in an effort to practice for such an event. There are many other examples where a VR simulation is preferable to a physical meeting between people. Thus, there is a need to create a VR environment that is as realistic as possible using simulated human characters having authentic and unpredictable behaviors that can change over time.

### SUMMARY OF THE INVENTION

In view of the foregoing, an object of this specification is to disclose a system in a virtual reality environment having a computer system, an interactive hardware device, where the interactive hardware device emulates an object in the virtual reality environment, and software residing in the computer system. The software includes an adaptive self-learning personality profile, based on at least one of a user's inputs to the interactive hardware device and a predetermined set of parameters and a character interface, where the character interface interacts with the user using the interactive hardware device based on the personality profile, where the personality profile is based at least in part on signals from the interactive hardware device, and where the personality profile generates responses to the user by sending signals to the interactive hardware device.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 illustrates a system block diagram of a virtual reality simulation using interactive hardware devices and software having a personality profile and using a humanoid character interface;

FIG. 2 illustrates a system block diagram of a computer system used to host a VR simulation environment;

FIG. 3 illustrates a computer program product in accordance with an embodiment that includes a computer-readable storage medium and program instructions;

FIG. 4 illustrates a group of interactive hardware devices used for the exchange of information between the user and the computer system; and

FIG. 5 illustrates a flow diagram of the software used to create a virtual reality simulation environment as disclosed herein.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

Disclosed is a system in a virtual reality environment that includes a computer system, an interactive hardware device, where the interactive hardware device emulates an object in the virtual reality environment, and software residing in the computer system, that includes an adaptive self-learning personality profile, based on at least one of a user's inputs to the interactive hardware device and a predetermined set of parameters; and a character interface, wherein the character interface interacts with the user using the interactive hardware device based on the personality profile, where the personality profile is based at least in part on signals from the interactive hardware device, and where the personality profile generates responses to the user by sending signals to the interactive hardware device.

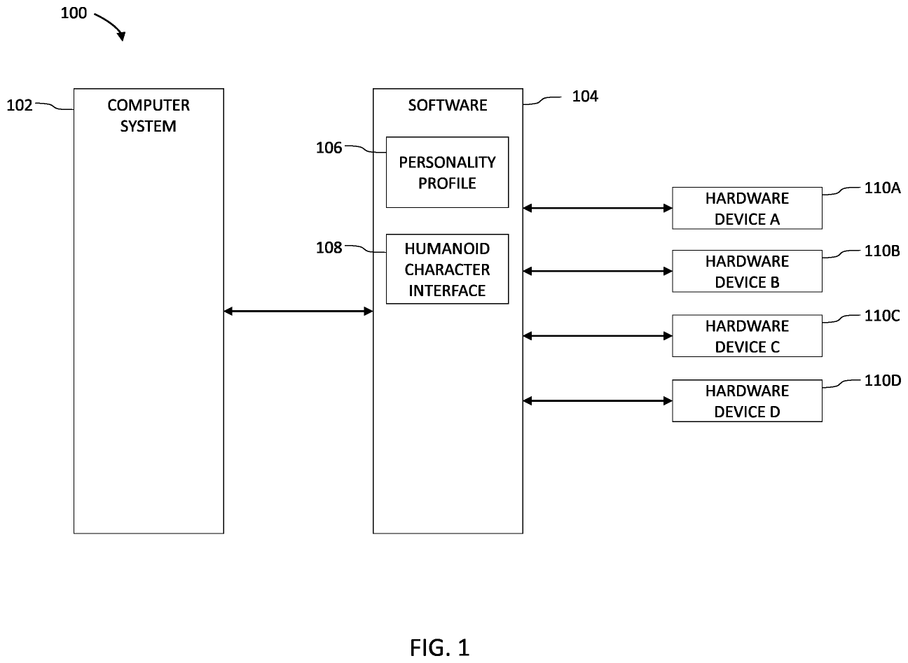

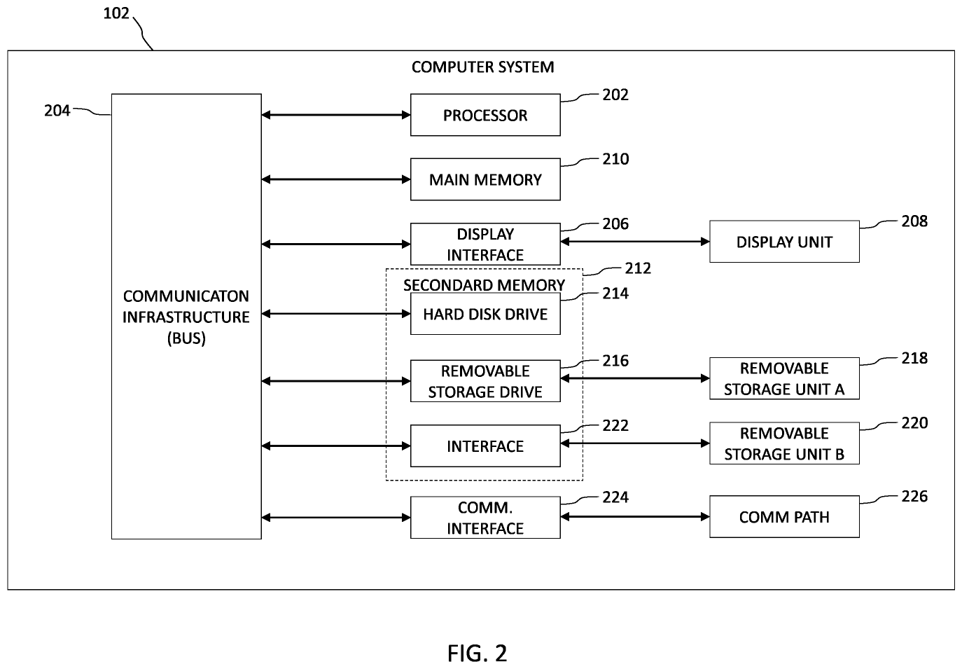

FIG. 1 illustrates a system block diagram 100 of a virtual reality simulation using interactive hardware devices 110A-110D. The system block diagram 100 includes a computer system 102, software 104, and one or more interactive hardware devices 110A-110D. The computer system 102 is fully described in FIG. 2 below. The software 104 is hosted on the computer system 102 and includes a personality profile 106 and a character interface 108 that emulates a human, humanoid, robotic, or animal character and is the primary interface for the user to communicate with the computer. According to some embodiments, the software 104 includes an operating system that runs on the computer system 102. In other embodiments, the software 104 resides in a remote computer or processor (not shown).

According to some embodiments, the character interface 108 emulates a human, robotic, machine interface, or animal character to better interface to the VR simulation environment requires. The personality profile 106 can be a pre-programmed profile of desirable traits the user wishes the interactive character to possess. In some embodiments, the personality profile 106 is configured by the user prior or during use of the VR simulation environment. In this manner, the personality profile 106 is edited, amended, or updated depending on the desires and needs of the user during their time in the VR simulation. In yet other embodiments, the personality profile 106 can be a self-learned and self-adaptable software program that learns from past events and user inputs and changes or otherwise updates itself as the user is in the simulation.

In yet other embodiments, the personality profile 106 can be configured to exhibit unwanted, uncommanded, or unusual traits. As an example, the personality profile of a patient undergoing a medical procedure exhibits hesitation during a medical procedure to simulate fear, pain, or discomfort, forcing the user to halt the intended procedure to attend to a new set of issues not anticipated by the user. In some embodiments, the VR simulation emulates a military or police action or an industrial setting. In another example, the personality profile in an adult entertainment simulation can express interest in trying new techniques not anticipated or requested by the user in an attempt to explore the user's interests, both conscious and subconscious.

According to some embodiments, the personality profile adapts to each user by modifying the response to the user's inputs via the interactive hardware devices 110A-110D. This is done to enable the VR simulation environment to better simulate a real human reaction via the character interface 108 by forming non-logical and undesirable traits in response to user inputs. As a result, the character interface 108 emulates a human character that can be hesitant, reluctant to act in a desired way, anti-social, self-destructive, and the like. In some embodiments, the character interface 108 acts contrary to specific commands from the user, just as in real life, people that interact with the user do not always cooperate or response exactly as the user wishes. This feature of the character interface 108 thus enhances the realism in a VR simulation by not acting logically, acting contrary to the user's desires, and can exhibit nonsensical and illogical actions and emotions in direct conflict with commands and preferences of the user.

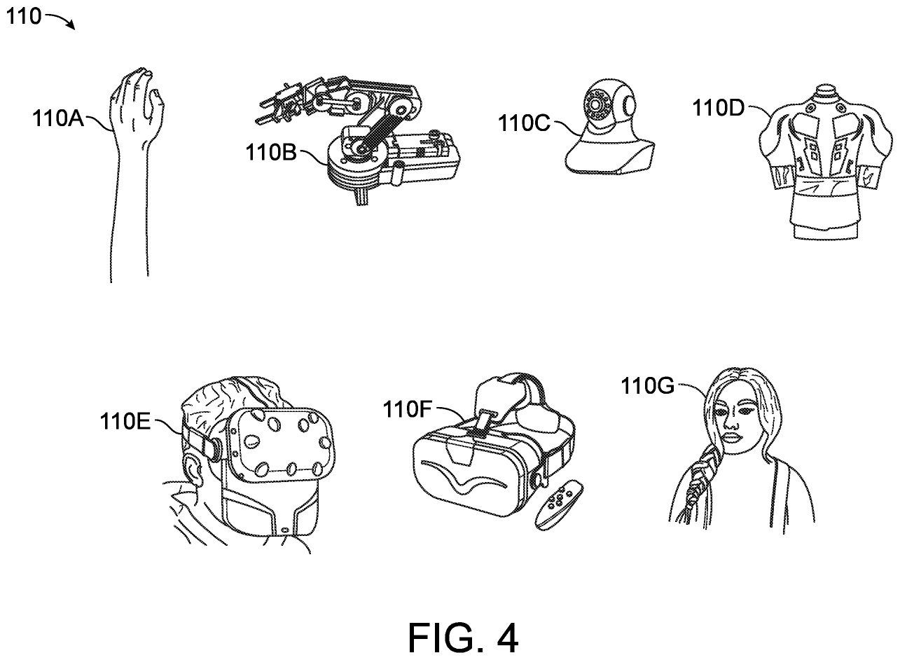

The interactive hardware devices 110A-110D are the primary input/output devices for communication between the user and the computer system 102. The interactive hardware devices 110A-110D emulate a tool, a human, an animal, a machine, or a material to enhance the user's experience in the VR environment. According to some embodiments, the interactive hardware devices 110A-110D receive signals from the computer system 102. In other embodiments, the interactive hardware devices 110A-110D sends signals to the computer system 102. In yet other embodiments, the interactive hardware devices 110A-110D both send and receive signals to and from the computer system 102. Examples of interactive hardware devices 110A-110D are fully disclosed in FIG. 4, below.

FIG. 2 illustrates a high-level block diagram of computer system 102, which can be used to implement one or more embodiments disclosed in this document. More specifically, computer system 102 can be used to implement hardware components of systems capable of performing methods described herein. Although one exemplary computer system 102 is shown, computer system 102 includes a communication path 226, which connects computer system 102 to additional systems (not depicted) and can include one or more wide area networks (WANs) and/or local area networks (LANs) such as the Internet, intranet(s), and/or wireless communication network(s). Computer system 102 and additional systems are in communication via communication path 226, e.g., to communicate data between them.

Computer system 102 includes one or more processors, such as processor 202. Processor 202 is connected to a communication infrastructure 204 (e.g., a communications bus, cross-over bar, or network). Computer system 102 can include a display interface 206 that forwards graphics, textual content, and other data from communication infrastructure 204 (or from a frame buffer, not shown) for display on a display unit 208. Computer system 102 also includes a main memory 210, preferably random access memory (RAM), and can also include a secondary memory 212. Secondary memory 212 can include, for example, a hard disk drive 214 and/or a removable storage drive 216, representing, for example, a floppy disk drive, a magnetic tape drive, or an optical disc drive. Hard disk drive 214 can be in the form of a solid state drive (SSD), a traditional magnetic disk drive, or a hybrid of the two. There also can be more than one hard disk drive 214 contained within secondary memory 212. Removable storage drive 216 reads from and/or writes to a removable storage unit A 218 in a manner well known to those having ordinary skill in the art. Removable storage unit A 218 represents, for example, a floppy disk, a compact disc, a magnetic tape, or an optical disc, etc. which is read by and written to by removable storage drive 216. As will be appreciated, removable storage unit A 218 includes a computer-readable medium having stored therein computer software and/or data.

In alternative embodiments, secondary memory 212 can include other similar means for allowing computer programs or other instructions to be loaded into the computer system 102. Such means can include, for example, a removable storage unit B 220 and an interface 222. Examples of such means can include a program package and package interface (such as that found in video game devices), a removable memory chip (such as an EPROM), secure digital card (SD card), compact flash card (CF card), universal serial bus (USB) memory, or PROM) and associated socket, and other removable storage units B 220 and interfaces 222 which allow software and data to be transferred from the removable storage unit B 220 to computer system 102.

Computer system 102 can also include a communications interface 224. Communications interface 224 allows software and data to be transferred between the computer system 102 and external devices. Examples of communications interface 224 can include a modem, a network interface (such as an Ethernet card), a communications port, or a PC card slot and card, a universal serial bus port (USB), and the like. Software and data transferred via communications interface 224 are in the form of signals that can be, for example, electronic, electromagnetic, optical, or other signals capable of being received by communications interface 224. These signals are provided to communications interface 224 via communication path (i.e., channel) 226. Communication path 226 carries signals and can be implemented using wire or cable, fiber optics, a phone line, a cellular phone link, an RF link, and/or other communications channels.

In the present description, the terms “computer program medium,” “computer usable medium,” and “computer-readable medium” are used to refer to media such as main memory 210 and secondary memory 212, removable storage drive 216, and a hard disk installed in hard disk drive 214. Computer programs (also called computer control logic) are stored in main memory 210 and/or secondary memory 212. Computer programs also can be received via communications interface 224. Such computer programs, when run, enable the computer system 102 to perform the features discussed herein. In particular, the computer programs, when run, enable processor 202 to perform the features of the computer system 102. Accordingly, such computer programs represent controllers of the computer system 102. Thus it can be seen from the forgoing detailed description that one or more embodiments provide technical benefits and advantages.

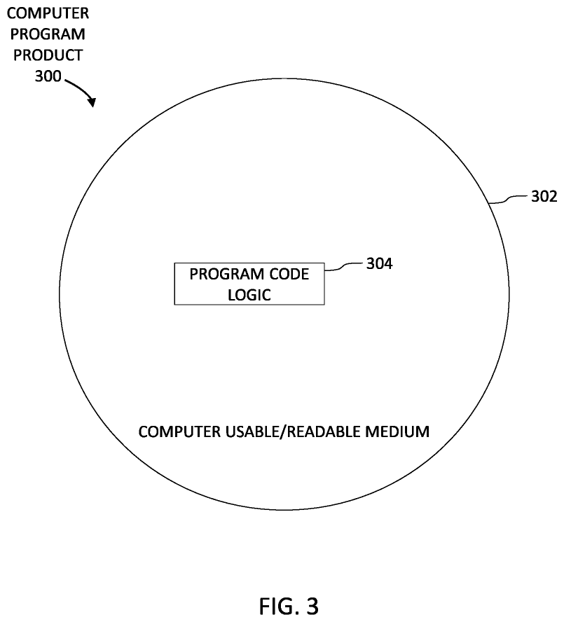

Referring now to FIG. 3, a computer program product 300 in accordance with an embodiment that includes a computer-readable storage medium 302 and program instructions 304 is generally shown.

Embodiments can be a system, a method, and/or a computer program product. The computer program product can include a computer-readable storage medium (or media) having computer-readable program instructions thereon for causing computer system 102 to carry out aspects of embodiments of the present invention.

The computer-readable storage medium can be a tangible device that can retain and store instructions for use by an instruction execution device. The computer-readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a wave-guide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out embodiments can include assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer-readable program instructions can execute entirely on the operator's computer, partly on the operator's computer, as a stand-alone software package, partly on the operator's computer and partly on a remote computer or entirely on the remote computer or server. In the latter scenario, the remote computer can be connected to the operator's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer-readable program instructions by utilizing state information of the computer-readable program instructions to personalize the electronic circuitry, in order to perform embodiments of the present invention.

According to one embodiment, the software 104 communicates with the interactive hardware devices 110A-110D to control direction, speed, movement, response time, duration, frequency, and vibration of the interactive hardware devices 110A-110D. The software 104 generates data packages that are triggered by some external action. The triggering action can be predetermined and stored in the personality profile 106, commanded by the user via the interactive hardware devices 110A-110D, occur randomly, or be a function of two or more of these items.

In one example, the user speaks into a microphone and says “speed up”. The software 104 will update the humanoid character interface 108 to react accordingly, and also will send a data packet to the interactive hardware devices 110A-110D to increase speed as appropriate.

According to some embodiments, signals to and from each interactive hardware device 110A-110D is processed as described herein. As originally designed, the interactive hardware devices 110A-110D includes an interface designed by an in-game humanoid character or graphically-designed computer animated object through commands via voice and in-VR player control mechanisms. This is in contrast to standard game hardware design approaches where an external controller controls the in-game character or object. In the present disclosure, the interactive hardware devices 110A-110D are, in effect, controlling the user and physical objects in the real world.

According to some embodiments, the interactive hardware devices 110A-110D can also be used to perform other functions such as turning on the computer system 102 once a user is detected, and without any user motion to activate the computer system 102. In one example, once a user sits in a chair, the interactive hardware devices 110A-110D turns on the VR environment and begins the simulation. Thus stated, the computer system 102 controls the character interface 108 without user input, instead of awaiting inputs form the user.

FIG. 4 illustrates a group of interactive hardware devices 110A-110G used for the exchange of information between the user and the computer system 102 as described herein. The devices include a prosthetic limb 110A for simulating a human arm or leg with realistic hand or foot action. According to some embodiments, the prosthetic limb 110A is a prosthetic hand and is able to reach out and interact with objects positioned around the user in the real world, such as picking up an apple, holding a camera and taking a picture, or shaking a hand of a user. Other embodiments are possible and are not limited by the examples described herein. Another example interactive hardware device is a robot-controlled tool 110B having a tool being manipulated by a mechanical arm or appendage. The robot-controlled tool 110B can be a surgical scalpel or wrench with other embodiments possible. Next, camera 110C can be a webcam, a digital camera, an infrared camera, and the like for creating images and videos of the user's environment. According to some embodiments, the camera 110C can track eye, head, arm, and body movements of the user and send that information to the computer system 102. In other embodiments, the camera 110C can operate as a security device, detecting a user's facial features and preventing unauthorized users access to the computer system 102. The interactive hardware devices can also include a force-feedback vest or body suit 110D that responds to commands from the computer system 102 and generates pressure, heat, simulated impacts, and the like to a user who wears the body suit 110D during interactive VR simulation sessions. According to some embodiments, the body suit 110D can encompass the chest area, the chest and arms, or the full body of the user to provide a fully immersive and physical response device for the user to wear.

According to some embodiments, another interactive hardware device is a scent mask 110E. The scent mask 110E is configured to create scents and smells based on signals from the computer system 102 to increase the simulation of an environment currently being generated by the computer system 102. In one example, a VR simulation of a repair on an oil pipeline can use the scent mask 110E to generate the scent of grease or oil in an effort to further convince the user that the simulation is as realistic as possible. In another example, the scent mask 110E can generate human smells such as sweat, body odor, and the like to emulate a simulated person under stress or during a sports event. Other embodiments are possible and are not limited by the examples disclosed herein. Virtual reality goggles 110F are visual devices for displaying to the user a simulated environment being generated by the computer system 102. Finally, another embodiment of the interactive hardware device is an adult entertainment doll 110G.

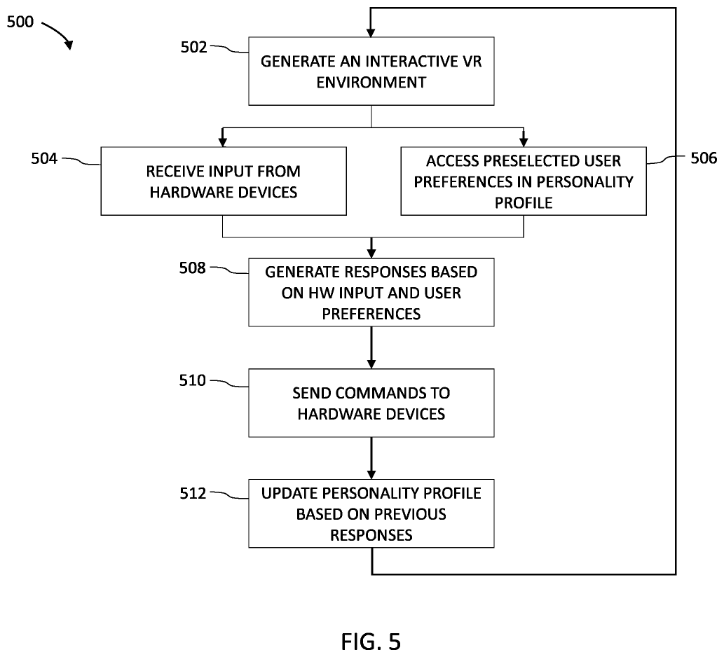

FIG. 5 illustrates a flow diagram 500 of the software 104 (of FIG. 1) used to create a virtual reality environment as disclosed herein. The flow diagram 500 includes generating an interactive virtual reality environment at block 502. Next, at block 504, the computer system 102 receives input from the one or more interactive hardware devices 110A-110D. The input can take the form of voice commands, mouse presses, joystick manipulation, force-feedback signals from wearable vests, tracking of head movement via a head- and eye-tracking device, and the like. Other embodiments are possible and are not limited by the examples mentioned in this disclosure. At the same time the computer system 102 is receiving input signals from the interactive hardware devices 110A-110D, the software 104 simultaneously accesses the personality profile 106 residing in main memory 210 or secondary memory 212 shown at block 506. From both inputs (the interactive hardware devices 110A-110D and the personality profile 106), the software 104 will select a response and send signals back to the interactive hardware devices 110A-110D at block 508. The software 104 will also generate audio and visual signals for the display unit 208 and sends those commands to the hardware devices at block 510 for use by the user. Finally, at block 512 the software 104 will update the personality profile 106 based on the set of responses already generated by the software 104 and the user's inputs to the computer system 102 through the interactive hardware devices 110A-110D.

According to some embodiments, disclosed herein is a system in a virtual reality environment that includes a computer system, an interactive hardware device, where the interactive hardware device emulates an object in the virtual reality environment, and software residing in the computer system, that includes an adaptive self-learning personality profile, based on at least one of a user's inputs to the interactive hardware device and a predetermined set of parameters; and a character interface, wherein the character interface interacts with the user using the interactive hardware device based on the personality profile, where the personality profile is based at least in part on signals from the interactive hardware device, and where the personality profile generates responses to the user by sending signals to the interactive hardware device.

The system may also include where the interactive hardware device is configured to emulate at least one of a tool, a human, an animal, a machine, and a material. The system may also include where the interactive hardware device is configured to emit smells corresponding to the virtual reality environment the user is in. The system may also include where the interactive hardware device is at least one of a vest and a body suit worn by the user, and is configured to apply pressure to locations on the user's body in response to signals generated by the personality profile. The system may also include where the interactive hardware device has a movable component for interacting with physical objects in proximity to the user. The system may also include a sensor for transferring images of the user's environment for use in the virtual reality environment. The system may also include where the virtual reality environment emulates at least one of a military or police action, a medical procedure, an underwater environment, a human interaction situation, an industrial environment, and an aerospace environment. The system may also include where the personality profile employs predictive modeling to generate a response to the user based at least in part on previous inputs by the user. The system may also include where the personality profile emulates non-logical characteristics despite user commands to the contrary, to include at least one of hesitation in response to user commands, physical resistance to user inputs, and a refusal to respond to user commands.

According to some embodiments, disclosed herein is a system in a virtual reality environment for adult entertainment that includes a computer system, an interactive hardware device, where the interactive hardware device emulates a human in the virtual reality environment, software residing in the computer system, having an adaptive self-learning personality profile, based on at least one of a user's inputs to the interactive hardware device and a predetermined set of parameters, and a humanoid character interface, wherein the humanoid character interface interacts with the user using the interactive hardware device based on the personality profile, where the personality profile is based at least in part on signals from the interactive hardware device, and where the personality profile generates responses to the user by sending signals to the interactive hardware device.

The system may also include where the interactive hardware device is configured to emulate a human body part. The system may also include where the interactive hardware device is configured to emit smells associated with activities in the virtual reality adult entertainment environment. The system may also include where the interactive hardware device is at least one of a vest and a body suit worn by the user, and is configured to apply pressure to locations on the user's body in response to signals generated by the personality profile. The system may also include where the interactive hardware device has a movable component for interacting with physical objects in proximity to the user. The system may also include a sensor for transferring images of the user's environment for use in the virtual reality environment. The system may also include where the personality profile employs predictive modeling to generate a response to the user based at least in part on previous inputs by the user. The system may also include where the personality profile emulates non-logical humanoid characteristics despite user commands to the contrary, to include at least one of hesitation in response to user commands, physical resistance to user inputs, and a refusal to respond to user commands.

According to some embodiments, disclosed herein is a method of treating a psychological, psychiatric, or medical disorder by providing a virtual reality experience to a user, that includes generating, by a computer system operating software, an interactive virtual environment using one or more visual, aural, and physical interactive hardware devices, receiving, by the computer system, signals from the user manipulating the interactive hardware devices, generating, by the computer system, commands to the interactive hardware devices based on a personality profile; and updating the personality profile based at least in part on the user's signals and a set of preselected preferences.

The method may also include where updating the personality profile includes adding human personality traits such as delaying responding to user commands, refusal to respond to user commands, and deception to make the virtual reality experience more realistic. According to some embodiments, the personality traits can also include pre-programmed action commands should the user prefer a personality that is completely predictable and shows no self-learning characteristics. The method may also include where the virtual reality environment is an adult entertainment environment.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

What is claimed is:

1. A system in a virtual reality environment, comprising:
a computer system;
an interactive hardware device, wherein the interactive hardware device emulates an object in the virtual reality environment; and
software residing in the computer system, comprising:
an adaptive self-learning personality profile, based on at least one of a user's inputs to the interactive hardware device and a predetermined set of parameters; and
a character interface, wherein the character interface interacts with the user using the interactive hardware device based on the personality profile, wherein
the personality profile is based at least in part on signals from the interactive hardware device, and wherein
the personality profile generates responses to the user by sending signals to the interactive hardware device.

2. The system of claim 1, wherein the interactive hardware device is configured to emulate at least one of a tool, a human, an animal, a machine, and a material.

3. The system of claim 1, wherein the interactive hardware device is configured to generate smells corresponding to the virtual reality environment the user is in.

4. The system of claim 1, wherein the interactive hardware device is at least one of a vest and a body suit worn by the user, and is configured to apply pressure to locations on the user's body in response to signals generated by the personality profile.

5. The system of claim 1, wherein the interactive hardware device further comprises a movable component for interacting with physical objects in proximity to the user.

6. The system of claim 1, further comprising a sensor for transferring images of the user's environment for use in the virtual reality environment.

7. The system of claim 1, wherein the virtual reality environment emulates at least one of a military or police action, a medical procedure, an underwater environment, a human interaction situation, an industrial environment, and an aerospace environment.

8. The system of claim 1, wherein the personality profile employs predictive modeling to generate a response to the user based at least in part on previous inputs by the user.

9. The system of claim 1, wherein the personality profile emulates non-logical characteristics despite user commands to the contrary, to include at least one of hesitation in response to user commands, physical resistance to user inputs, and a refusal to respond to user commands.

10. A system in a virtual reality environment for adult entertainment, comprising:
a computer system;
an interactive hardware device, wherein the interactive hardware device emulates a human in the virtual reality environment;
software residing in the computer system, comprising:
an adaptive self-learning personality profile, based on at least one of a user's inputs to the interactive hardware device and a predetermined set of parameters; and
a humanoid character interface, wherein the humanoid character interface interacts with the user using the interactive hardware device based on the personality profile, wherein
the personality profile is based at least in part on signals from the interactive hardware device, and wherein
the personality profile generates responses to the user by sending signals to the interactive hardware device.

11. The system of claim 10, wherein the interactive hardware device is configured to emulate a human body part.

12. The system of claim 10, wherein the interactive hardware device is configured to generate smells associated with activities in the virtual reality adult entertainment environment.

13. The system of claim 10, wherein the interactive hardware device is at least one of a vest and a body suit worn by the user, and is configured to apply pressure to locations on the user's body in response to signals generated by the personality profile.

14. The system of claim 10, wherein the interactive hardware device further comprises a movable component for interacting with physical objects in proximity to the user.

15. The system of claim 10, further comprising a sensor for transferring images of the user's environment for use in the virtual reality environment.

16. The system of claim 10, wherein the personality profile employs predictive modeling to generate a response to the user based at least in part on previous inputs by the user.

17. The system of claim 10, wherein the personality profile emulates non-logical humanoid characteristics despite user commands to the contrary, to include at least one of hesitation in response to user commands, physical resistance to user inputs, and a refusal to respond to user commands.

18. A method of treating a psychological, psychiatric, or medical disorder by providing a virtual reality experience to a user, comprising:
generating, by a computer system operating software, an interactive virtual environment using one or more visual, aural, and physical interactive hardware devices;
receiving, by the computer system, signals from the user manipulating the interactive hardware devices;
generating, by the computer system, commands to the interactive hardware devices based on a personality profile; and
updating the personality profile based at least in part on signals from the user and a set of preselected preferences.

19. The method of claim 18, wherein updating the personality profile includes adding human personality traits such as delaying responding to user commands, refusal to respond to user commands, and deception to make the virtual reality experience more realistic.

20. The method of claim 18, wherein the virtual reality environment is an adult entertainment environment.
